(19) United States
(12) Patent Application Publication
(10) Pub. No.: US 2011/0152358 A1
(43) Pub. Date: Jun. 23, 2011

(54) Compositions and Methods for Diagnosis and Treatment of Pancreatic Ductal Cancer

(76) Inventor: Murray Korc, Hanover, NH (US)

(21) Appl. No.: 13/059,088

(22) PCT Filed: Aug. 7, 2009
(86) PCT No.: PCT/US09/53094

§ 371 Date: Feb. 15, 2011

Related U.S. Application Data
(60) Provisional application No. 61/090,301, filed on Aug. 20, 2008.

(51) Int. Cl. A61K 31/7088; G01N 33/53; A61P 35/00

(52) U.S. Cl. 514/44 A; 435/7.1

(57) ABSTRACT

The present invention includes compositions and methods for diagnosing and treating pancreatic cancer. These compositions and methods are based on the finding that 14-3-3σ protein is secreted from pancreatic cancer cells and is therefore a specific biomarker protein.

### INTRODUCTION

This invention was made in the course of research sponsored by the National Institutes of Health, National Cancer Institute, under contract number CA-10130. The U.S. Government has certain rights in this invention.

### BACKGROUND OF THE INVENTION

Pancreatic ductal adenocarcinoma (PDAC) is the fourth leading cause of cancer mortality in the United States, with a five-year survival rate that remains under 5% (Jemal, A. et al. 2008. CA Cancer J. Clin. 58:71-96). Although our understanding of the molecular and genetic basis for this disorder is expanding, there has been only modest progress in its treatment. There is a high frequency of K-ras, p53, p16, and Smad4 mutations in PDAC, in conjunction with overexpression of tyrosine kinase receptors and their ligands and excessive activation of mitogenic signaling pathways (Bardeesy, N. and R. A. DePinho. 2002. Nat. Rev. Cancer 2:897-909; Korc, M. 1998. Surg. Oncol. Clin. N. Am. 7:25-41). Moreover, the cancer cells in PDAC exhibit apoptosis resistance. The mechanisms that underlie this increased resistance to apoptosis-inducing signals have not been completely elucidated, but have been attributed to constitutive NFKB and Akt activation, altered expression of anti-apoptotic proteins such as Bcl-2, Bcl-X_{L}, and the inhibitor of apoptosis proteins (IAP), and increased Smad7 and thioredoxin expression (Westphal, S. and H. Kalthoff. 2003. Mol. Cancer 2:6; Arnold, N. B. et al. 2004. Cancer Res. 64:3599-3606).

The 14-3-3 family consists of small (28-33 kDa) acidic proteins with evolutionarily conserved amino acid sequences that participate in the regulation of cell proliferation and survival. There are seven distinct mammalian isoforms of 14-3-3 (β, ε, γ, η, σ, θ/τ, and ζ) which often form hetero or homodimers and bind to more than 100 different proteins (Fu, H. et al. 2000. Ann. Rev. Pharmacol. Toxicol. 40:617-647; Rubio, M. P. et al. 2004. Biochem. J. 379:395-408). Most commonly, 14-3-3 proteins bind to target proteins possessing phosphoserine and phosphothreonine motifs (RSxpSxP or RxY/FxpSxP; x denotes any amino acid and pS represents a phosphorylated serine) (Rubio, M. P. et al. 2004. Biochem. J. 379:395-408; Muslin, A. J. et al. 1996. Cell 84:889-897). In addition, some 14-3-3-binding partners exhibit variations from these motifs, and others lack these motifs and bind to 14-3-3 in a phosphorylation independent manner (Hermeking, H. 2003. Nat. Rev. Cancer 3:931-943).

The expression of 14-3-3□, which is also known as human mammary epithelial marker 1 (HME1), or stratifin, is mostly restricted to epithelial cells, and is known to be altered in several human cancers (Hermeking, H. 2003. Nat. Rev. Cancer 3:931-943). 14-3-3σ is down-regulated in many human cancers, where it has been proposed to function as a tumor suppressor gene. In breast cancer and hepatocellular carcinoma, for instance, 14-3-3σ levels are significantly decreased, principally due to silencing of the gene through hypermethylation (Ferguson, A. T. et al. 2000. Proc. Natl. Acad. Sci. USA 97:6049-6054; Iwata, N. et al. 2000. Oncogene 19:5298-5302). By contrast, 14-3-3σ expression is increased in PDAC (Friess, H. et al. 2003. Cell. Mol. Life Sci. 60:1180-1199; Logsdon, C. D. et al. 2003. Cancer Res. 63:2649-2657; Nakamura, T. et al. 2004. Oncogene 23:2385-2400; Iacobuzio-Donahue, C. A. et al. 2003. Cancer Res. 63:8614-8622; Adsay, N. V. et al. 2005. Semin. Radiat. Oncol. 15:254-264; Justinix, S. R. et al. 2005. Cancer Biol. Ther. 4:596-601), as well as in other cancers, such as colon carcinoma, and head and neck squamous cell carcinomas (Perathoner, A. et al. 2005. Clin. Cancer Res. 11:3274-3279; Villaret, D. B. et al. 2000. Laryngoscope 110:374-381).

The specific biological role of the elevated expression of 14-3-3σ in any human cancer is not yet known. However, several papers have reported on results of experiments attempting to elucidate the mechanism. Guweidhi et al. (2004. Carcinogenesis 25:1575-1585) teach that the expression of 14-3-3σ is enhanced in pancreatic cancer and that the gene, together with other genetic and epigenetic alterations of potential partners to 14-3-3σ activity may be important in carcinogenesis. The authors specifically report that unlike the role of 14-3-3σ in other circumstances where its function has been linked to sustaining of a G2 checkpoint in the cell cycle and anti-apoptotic activity, those functions of 14-3-3σ do not appear to operate in pancreatic cancer.

Research has also examined a role for 14-3-3σ in the diagnosis of different forms of cancer. In a study exploring the role of various genes in biliary carcinoma, it was reported that markers other than 14-3-3σ were most useful, although the paper does report that tissue microarray methods can be used to monitor markers for pancreatic cancer (Swierczynski, S. L. et al. 2004. Hum. Pathol. 35:357-366). U.S. Publication No. 2005/0009067 discloses that pancreatic cancer diagnosis can involve monitoring the expression of at least two genes at once, where the 14-3-3σ gene is one of the genes that can be used in the diagnostic methods claimed in the patent. The patent application also discusses use of a kit based on detection of polypeptides encoded by two or more genes through binding to antibodies and that one of the polypeptides to be monitored could include 14-3-3σ. U.S. Publication No. 2007/0212738 also discloses diagnostic methods involving 14-3-3σ but these are directed at predicting the effectiveness of cancer treatment when the treatment involves use of an EGFR kinase inhibitor. The methods are based on assessing the expression of epithelial biomarkers in tumor cells and one of the markers discussed is 14-3-3σ.

There are currently no sensitive diagnostic markers for PDAC that allow for is early detection or for monitoring the effectiveness of certain types of treatment and recurrence of the cancer.

### SUMMARY OF THE INVENTION

The present invention features a composition for identifying patients with pancreatic cancer or patients with recurrence of pancreatic cancer. This composition comprises an agent that binds a pancreatic cancer-specific biomarker protein. In particular embodiments, the agent is used in a method of diagnosing pancreatic cancer by contacting a test sample from a human subject with the agent and detecting the level of binding of the test agent to the pancreatic cancer-specific biomarker protein, wherein an increase in the level of binding as compared to a level known to be seen in patients without pancreatic cancer is indicative of the presence of pancreatic cancer. In a preferred embodiment the pancreatic cancer specific biomarker protein is 14-3-3σ.

Yet another object of the present invention is a method of diagnosing pancreatic cancer in a patient which comprises contacting blood from a patient suspected of having pancreatic cancer or a patient that previously was diagnosed with pancreatic cancer in vitro with an agent that binds the pancreatic cancer-specific biomarker protein. In a preferred embodiment the pancreatic cancer-specific protein is 14-3-3σ.

Yet another object of the present invention is a method for treating pancreatic cancer in a patient which comprises administering to a patient with pancreatic cancer an effective amount an agent that inhibits activity of EGFR and an agent that inhibits activity of 14-3-3σ.

### BRIEF DESCRIPTION OF THE DRAWINGS

FIG. 1 depicts the expression of 14-3-3 isoforms in pancreatic tissue and pancreatic cancer cell lines. In FIG. 1A, 14-3-3 mRNA expression in laser captured pancreatic tissue is depicted. RNA isolated from laser captured normal ductal cells from 3 normal pancreata and cancer cells from 7 PDAC samples was subjected to real-time quantitative PCR using isoform specific primers and probes. Data are the means ±SE. *, p<0.03 when compared with values obtained in normal ducts, or with values in the cancer cells for the other members of the 14-3-3 family. In FIG. 1B, 14-3-3 mRNA expression in pancreatic cancer cell lines is depicted. Total RNA isolated from the indicated cells lines was reverse transcribed, and subjected to real-time PCR as described in the materials and methods section. Results were normalized to 18S levels. Data are the means of two independent experiments performed in duplicate in which similar results were obtained.

FIG. 2 depicts the effects of 14-3-3σ overexpression on PANC-1 cell apoptosis and motility. In FIG. 2B, the effect of 14-3-3σ on survival is depicted. Sham transfected (Sham) and 14-3-3σ expressing clones (C1 and C7) were incubated for 48 hours with 10 μg/mL of cisplatinum. MTT assays were performed. Data are the means ±SE of three independent experiments performed with 6 wells per sample. *, p<0.01 when compared with Sham. FIG. 2B depicts the effects of the Src-kinase inhibitor PP2 on motility. Sham and 14-3-3σ expressing clones C1 and C7 were incubated in the absence (SF) or presence of the indicated additions. SF, serum free media only; EGF, 1 nmol/L EGF added to serum free media; EGF+PP2, 1 nmol/L EGF and 10 μmol/L PP2 added to serum free media. Data are the means +SE from three independent experiments. Data were analyzed by one-way analysis of variance (ANOVA) followed by Tukey-HSD.*, p<0.05 compared with sham SF; ‡, p<0.01 compared with Sham SF; **, p<0.01 compared with Sham EGF; #, p<0.05 compared with respective SF or EGF treatment).

FIG. 3 depicts the effects of 14-3-3σ on EGF-stimulated invasion. In FIG. 3A, the results are shown where the effects of EGF on invasion of sham transfected (Sham) and 14-3-3σ expressing clones (C1 and C7) were quantified. The number of invading cells in each membrane was counted using an inverted microscope (magnification, 200×) after staining with toluidine blue. Data are the means ±SE of duplicate determinations from 3 independent experiments. *, p<0.01 when compared with the EGF treated Sham. In FIG. 3B, the effects of inhibiting PI3K, p38 MAPK, and Src kinase on EGF-induced invasion in 14-3-3σ overexpressing cells (clone C1) are depicted. Cells were incubated for 20 hours in the absence (SF) or presence of EGF (1 nmol/L) alone, or EGF together with LY 294002 (5 μmol/L) or SB 203580 (10 μmol/L) or PP2 (10 μmol/L). Data are the means ±SE of duplicate determinations from 3 independent experiments. *, p<0.01 when compared with control values in the absence of EGF; **, p<0.01 when compared with EGF stimulation in the absence of any of the inhibitors.

FIG. 4 depicts the effects of silencing 14-3-3σ on motility and invasion in T3M4 cells. In FIG. 4A, results of wound-healing assays in 14-3-3σ silenced T3M4 cells are shown. Control shRNA (shCtrl) or 14-3-3σ specific shRNA (sh1 or sh2) infected cells were subjected to wound-healing assay in the absence (SF) or presence of EGF (1 nmol/L) with or without Src-kinase inhibitor PP2 for 24 hours. Data are the means ±SE from three independent experiments. *, p<0.01 compared with respective SF conditions; #, p<0.01 compared with respective EGF treatment. In FIG. 4B, the results gathered from matrigel invasion assays are shown. Control shRNA (shCtrl) or 14-3-3σ specific shRNA (sh2) infected cells (5×10⁴ cells/well) were subjected to matrigel invasion assay in the absence (SF) or presence of EGF (1 nmol/L) for 18 hours. For inhibition studies, sh2 infected cells were incubated in matrigel chambers either in the absence (SF) or presence of EGF (1 nmol/L) together with AG1517 (1 μmol/L) or PP2 (10 μmol/L). Data are means ±SE of duplicate determinations from three independent experiments. *, p<0.01 compared with EGF in shCtrl. **, p<0.01 compared with EGF in sh2. In FIG. 4C, the effects of 14-3-3σ overexpression on IGF-1 stimulated invasion are shown. Sham transfected (Sham) and 14-3-3σ expressing clones C1 and C7 (3×10⁴ cells/well) were plated on the upper wells of matrigel chambers and incubated in the absence (SF) or presence of IGF-1 (1 nmol/L) in the lower compartment as chemoattractant for 20 hours. Cells that had invaded through the matrigel layer were fixed and stained as described in the materials and methods section. Data are the means ±SE of duplicate determinations from 3 independent experiments. *, p<0.01 compared with IGF-1 treated Sham. D, effects of silencing 14-3-3σ on IGF-1 stimulated invasion. Control shRNA (shCtrl) or 14-3-3σ specific shRNA (sh1 or sh2) infected cells (5×10⁴ cells/well) were subjected to invasion assay in the absence (SF) or presence of IGF-1 (1 nmol/L) as chemoattractant for 18 hours. Data are the means ±SE of duplicate determinations from 3 independent experiments. *, p<0.01 compared to shCtrl SF; **, p<0.01 compared with IGF-1 treated shCtrl.

### DETAILED DESCRIPTION OF THE INVENTION

It has been found that 14-3-3σ enhances apoptosis resistance in pancreatic cancer cells and increases the invasiveness in response to EGF and IGF-1. Therefore, the present invention is method for treating PDAC wherein treatments aimed at suppressing function of 14-3-3σ are combined with EGFR-targeted therapy in order to enhance the therapeutic benefit to the patient. It has also been found that pancreatic cancer cells release 14-3-3σ and as such the present invention is a diagnostic method for PDAC wherein monitoring levels of 14-3-3σ protein in blood. An increase in the levels of 14-3-3σ protein in blood would correlate with either the presence of new disease or the recurrence of disease in patients that had previously been treated for PDAC.

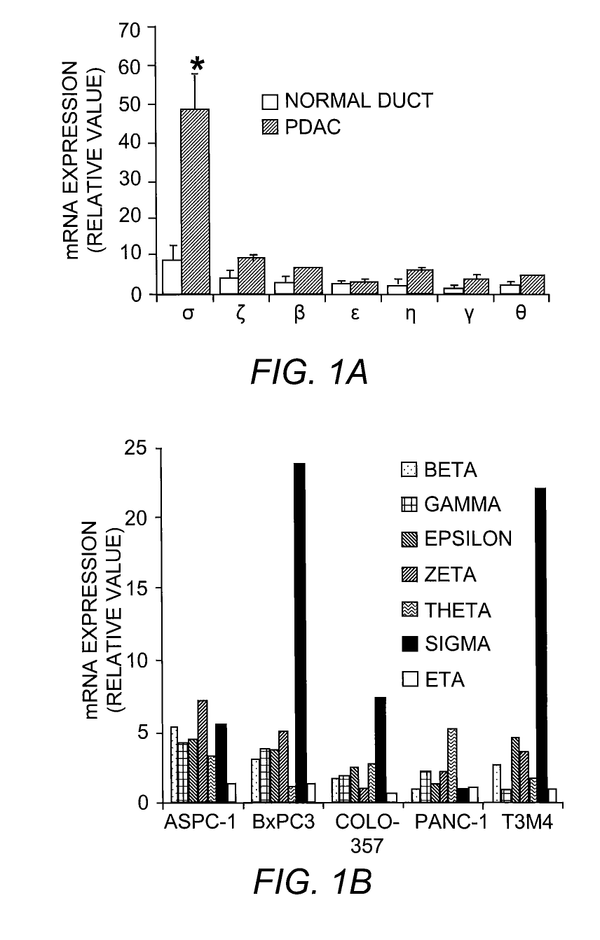

Experiments were performed to examine the underlying mechanism(s) involved in 14-3-3 expression and pancreatic carcinogenesis. The first step was to analyze 14-3-3 expression in human pancreatic tissue and pancreatic cancer cell lines. The expression of all seven 14-3-3 isoforms was determined in RNA prepared from laser-captured normal pancreatic duct cells and pancreatic cancer cells. Among all the 14-3-3 isoforms expressed in the laser-captured cancer cells, 14-3-3σ mRNA levels were the highest. Moreover, 14-3-3σ mRNA levels were increased 5-fold in cancer cells in comparison with the corresponding levels in RNA isolated from laser-captured normal ductal cells (FIG. 1A). All seven tested samples exhibited increased 14-3-3σ immunoreactivity in the cancer cells within the PDAC samples.

Analysis of the expression of all seven isoforms in five human pancreatic cancer cell lines by quantitative-PCR and immunoblotting revealed that all seven isoforms were expressed in all the cell lines, at both the mRNA (FIG. 1B) and protein level. High levels of 14-3-3σ mRNA were evident in BxPC3 and T3M4 cells, intermediate levels were present in ASPC-1 and COLO-357 cells, and low levels were present in PANC-1 cells. BxPC3 and T3M4 cells, which expressed high levels of 14-3-3σ mRNA also exhibited high levels of 14-3-3σ protein in both cell lysates and conditioned media. COLO-357 cells, which expressed intermediate levels of 14-3-3σ mRNA exhibited high intracellular levels of 14-3-3σ protein but low levels of the protein in the medium indicating that the high intracellular levels were due, in part, to attenuated release of 14-3-3σ protein. ASPC-1 and PANC-1 cells, which expressed low levels of 14-3-3σ mRNA, also expressed low levels of the protein. Furthermore, PANC-1 cells released low levels 14-3-3σ into conditioned media, whereas ASPC-1 cells did not release detectable levels of 14-3-3σ. This failure to release 14-3-3 may explain why ASPC-1 cells exhibited higher levels of intracellular 14-3-3σ than PANC-1 cells. In order to confirm that the presence of 14-3-3σ protein in the media was not simply due to its release from dead cells, the membrane was blotted for tubulin, a cytoplasmic protein. Tubulin was detected only in the whole cell lysates and not in the media from any of the cell lines. These data provided evidence for the role of 14-3-3 protein as a marker for pancreatic cancer.

The protein levels of 14-3-3θ were highest in PANC-1 cells and lowest in BxPC3 cells, and the levels of 14-3-3γ were highest in T3M4 cells and lowest in ASPC-1 cells. The protein levels for the remaining isoforms were relatively uniform in all five cell lines.

In view of the fact that PANC-1 cells expressed a low level of 14-3-3σ protein, it was believed that the cell line would be useful for assessing the biological role of 14-3-3σ in pancreatic cancer. Accordingly, PANC-1 clones (C1 and C7) that overexpressed 14-3-3σ were generated. 14-3-3σ levels in clones C1 and C7 were comparable to the endogenous expression levels in BxPC3 and COLO-357 cells and were much higher than the endogenous levels in PANC-1 cells. Overexpression of 14-3-3σ was also associated with its increased release into conditioned medium. Following a more prolonged exposure of the membrane, a faint band corresponding to 14-3-3σ was seen in conditioned medium from sham-transfected cells, in agreement with the observation that PANC-1 cells express low levels of endogenous 14-3-3σ. By immunofluorescence using an anti-14-3-3σ antibody, the overexpressed protein localized to both the cytoplasm and the peri-nuclear region. Similar results were observed with the anti-HA antibody.

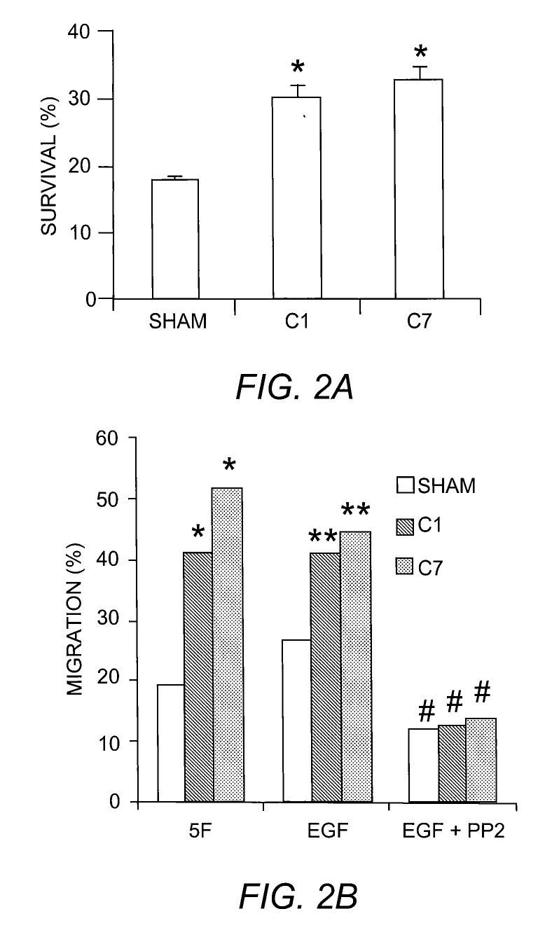

To determine whether high levels of 14-3-3σ modulate apoptotic signaling, the effects of cisplatinum on PARP cleavage and caspase-3 activation were examined. In response to 10 μg/mL cisplatinum, both 14-3-3σ overexpressing clones exhibited attenuated PARP cleavage and markedly decreased generation of cleaved caspase-3, which is the activated form of caspase-3, by comparison with the effects seen in the sham transfected PANC-1 cells. This decrease in cisplatinum-induced apoptosis was associated with a significant (p<0.01) increase in the survival of these clones, as determined with an MTT assay (FIG. 2A).

To determine whether high levels of 14-3-3σ were associated with alteration in migration and invasion properties, migration and invasion assays were performed. Given the importance of the EGF receptor in the aggressiveness of pancreatic cancer and its known capacity for stimulating cell migration and invasion (Korc, M. 1998. Surg. Oncol. Clin. N. Am. 7:25-41), the effects of EGF on motility were also studied.

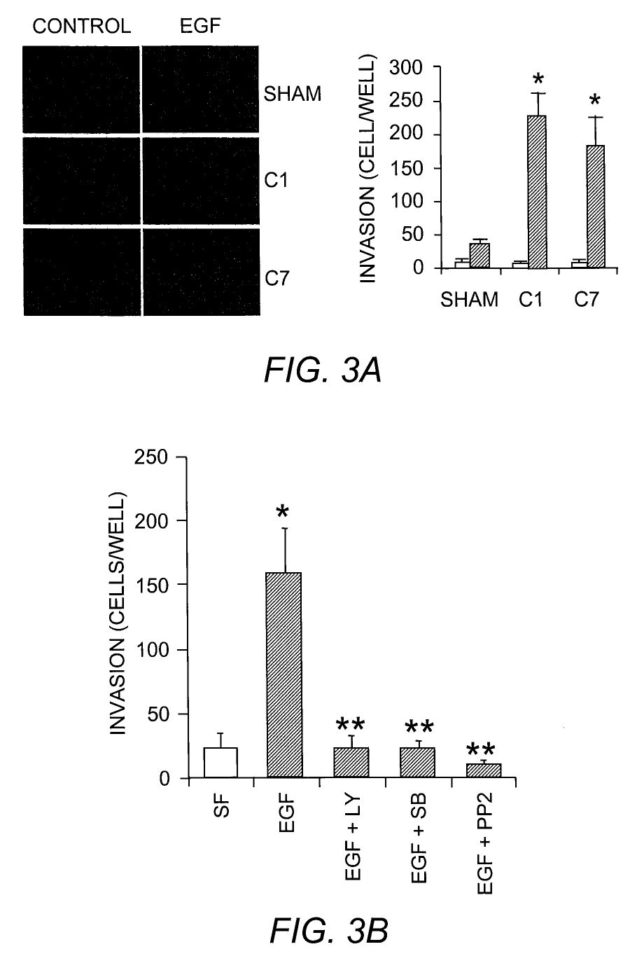

14-3-3σ overexpressing clones were twice as motile as sham cells. EGF caused a slight but significant increase in the motility of sham-transfected cells (FIG. 2B), but did not cause an additional increase in motility in 14-3-3σ overexpressing cells (FIG. 2B). The Src-kinase inhibitor PP2 significantly attenuated basal, EGF-stimulated (FIG. 2B), and 14-3-3σ enhanced motility (FIG. 2B). By contrast, while there was a trend for EGF to enhance the invasion of sham transfected cells, this effect was not statistically significant, whereas EGF markedly enhanced the invasion of 14-3-3σ overexpressing clones (FIG. 3A). LY294002, a PI3 kinase inhibitor, SB203580, a p38 mitogen-activated protein kinase (p38MAPK) inhibitor, and PP2, the Src-kinase inhibitor, all suppressed EGF-mediated invasion in 14-3-3σ overexpressing clones (FIG. 3B).

To determine whether silencing endogenously high levels of 14-3-3σ would make pancreatic cancer cells more sensitive towards cisplatinum, the lentiviral shRNA approach was used to downregulate 14-3-3σ gene in T3M4 cells, which have high endogenous levels of 14-3-3σ. Two different shRNA sequences (sh1 and sh2) targeting 14-3-3σ were determined to reduce 14-3-3a levels, with sh2 mediated knockdown being more effective than sh1. By contrast, the scrambled control shRNA (shCtrl) did not alter 14-3-3σ protein levels, and the shRNA specific to 14-3-3σ had no effect on the mRNA and protein levels of other members of 14-3-3 family.

Next, T3M4 cells infected with either control or specific shRNA were incubated with cisplatinum (10 νg/mL) for 2, 6, or 24 hours, and apoptosis was assessed by immunoblotting for cleaved PARP and cleaved caspase-3. Cells with silenced 14-3-3σ exhibited increased apoptosis compared to non-silenced cells as evidenced by increased PARP cleavage and caspase-3 activation. Cells infected with sh2, which was better at silencing 14-3-3σ compared to sh1, were relatively more sensitive to cisplatinum as evidenced by higher levels of PARP cleavage and caspase-3 activation, and both were significantly more sensitive than control cells.

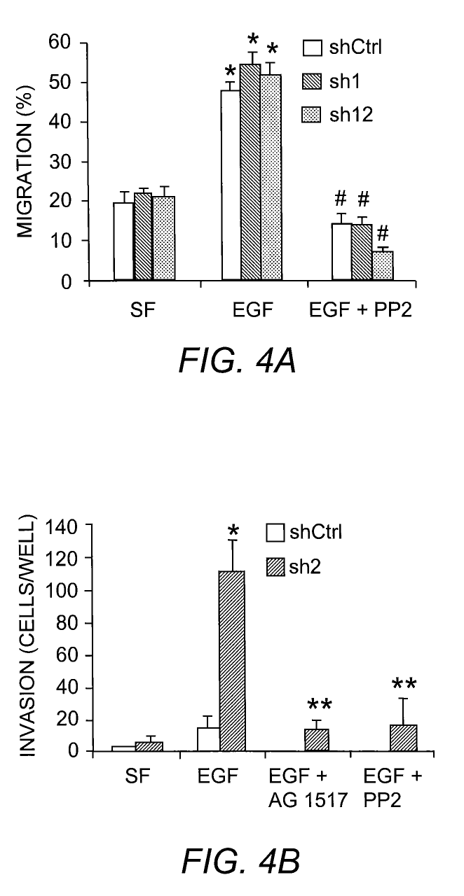

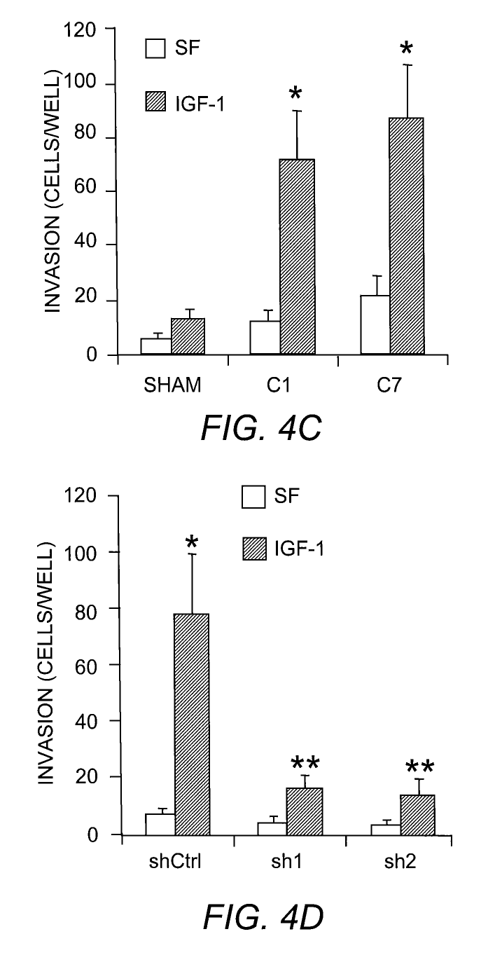

Silencing of 14-3-3σ did not lead to alterations in basal or EGF stimulated motility in T3M4 cells. Thus, cells infected with control shRNA and specific shRNA (sh1 or sh2) were equally motile (20%), and EGF stimulated the motility to about 50% in all three groups (FIG. 4). The EGF stimulated increase in motility was abrogated by Src-kinase inhibitor PP2. EGF induced a marked increase in invasion in 14-3-3σ downregulated cells (sh2 infected) as compared to invasion in control shRNA infected cells, and this stimulatory effect was suppressed by AG 1517 (1 μmol/L), an EGFR kinase inhibitor, and by PP2, a Src-kinase inhibitor (FIG. 4B).

In view of the divergent effects of 14-3-3σ levels in PANC-1 and T3M4 cells in relation to EGF-mediated cell invasion, the effects of IGF-1 on cell invasion in both cell lines were examined. IGF-1 significantly increased the invasion of PANC-1 clones overexpressing 14-3-3σ compared to its effect in sham cells (FIG. 4C). Conversely, the effects of IGF-1 on invasion were markedly attenuated in T3M4 cells in which 14-3-3σ was silenced (FIG. 4D).

Several lines of evidence suggest that 14-3-3σ may function as a tumor suppressor gene. First, 14-3-3σ is the only member of the 14-3-3 family that is induced following DNA damage (Samuel, T. et al. 2001. J. Biol. Chem. 276:45201-45206) and is required to prevent mitotic catastrophe following such damage (Chan, T. A. et al. 1999. Nature 401:616-620). Second, the ectopic expression of 14-3-3σ leads to a G2 arrest (Hermeking, H. et al. 1997. Mol. Cell 1:3-11), due to the sequestration of CDC2-cyclinB1 in the cytoplasm (Chan, T. A. et al. 1999. Nature 401:616-620). Third, 14-3-3σ is silenced or under-expressed in breast (Ferguson, A. T. et al. 2000. Proc. Natl. Acad. Sci. USA 97:6049-6054), prostate (Cheng, L. et al. 2004. Clin. Cancer Res. 10:3064-3068), and hepatic cancers (Iwata, N. et al. 2000. Oncogene 19:5298-5302). In these cases, there is strong evidence for epigenetic inactivation which occurs as a consequence of hypermethylation at the gene locus (Ferguson, A. T. et al. 2000. Proc. Natl. Acad. Sci. USA 97:6049-6054; Iwata, N. et al. 2000. Oncogene 19:5298-5302; Lodygin, D. et al. 2004. Oncogene 23:9034-9041). Additional mechanisms for the down-regulation of 14-3-3σ levels include mutations or functional deregulation of p53 which is known to directly induce 14-3-3σ expression (Hermeking, H. et al. 1997. Mol. Cell 1:3-11), and E3 ubiquitin ligase mediated degradation of 14-3-3σ (Urano, T. et al. 2002. Nature 417:871-875). Although the p53 gene is frequently mutated in PDAC, 14-3-3σ is overexpressed in this cancer (Friess, H. et al. 2003. Cell Mol. Life Sci. 60:1180-1199; Logsdon, C. D. et al. 2003. Cancer Res. 63:2649-2457; Nakamura, T. et al. 2004. Oncogene 23:2385-2400; Iacobuzio-Donahue, C. A. et al. 2003. Cancer Res. 63:8614-8622). In the cell lines investigated in this study, only COLO-357 cells have wild type p53 while the other four cell lines have non-functional p53 (Guweidhi, A. et al. 2004. Carcinogenesis 25:1575-1585; Seki, T. et al. 2001. Anticancer Res. 21:1919-1924). There was no correlation between p53 gene status and 14-3-3σ levels. Although the mechanisms underlying overexpression of 14-3-3σ in PDAC are not known, in contrast to several cancers where the 14-3-3σ gene is silenced by hypermethylation, the 14-3-3σ gene is hypomethylated in PDAC (Sato, N. et al. 2003. Cancer Res. 63:4158-4166).

The 14-3-3 family proteins regulate diverse cellular processes, often by binding to phosphorylated sites in many target proteins. Recently, 14-3-3σ was shown to preferentially form homodimer and this structural feature may be responsible for its unique role in response to DNA damage and human oncogenesis (Wilker, E. W. et al. 2005. J. Biol. Chem. 280:18891-18898). In addition, 14-3-3σ exerts anti-apoptotic actions, in part, due to its ability to sequester pro-apoptotic proteins such as BAD and BAX (Samuel, T. et al. 2001. J. Biol. Chem. 276:45201-45206; Zha, J. et al. 1996. Cell 87:619-628; Nomura, M. et al. 2003. J. Biol. Chem. 278:2058-2065). Thus, phosphorylation-dependent association of BAD, and phosphorylation-independent association of BAX with 14-3-3σ prevents them from translocating to the mitochondria and activating the downstream apoptotic cascade.

It has now been found that using laser captured samples that among all the members of the 14-3-3 family only 14-3-3σ is overexpressed in the pancreatic cancer cells. Furthermore, a comparison of the expression levels of all 14-3-3 family members in several pancreatic cancer cell lines showed that 14-3-3σ protein levels were relatively high in BxPC3, COLO-357, and T3M4, and relatively low in PANC-1 and ASPC-1 cells. This relative expression pattern for 14-3-3σ is in agreement with a previous report detailing 14-3-3σ expression in pancreatic cancer cells (Guweidhi, A. et al. 2004. Carcinogenesis 25:1575-1585). Therefore, detection of increased levels of 14-3-3σ protein are a biomarker for pancreatic cancer, and can be used to diagnose cancer in patients suspected of having the disease.

Given that PANC-1 cells expressed relatively low levels of endogenous 14-3-3σ at both the mRNA and protein levels, the experiments discussed above were performed where 14-3-3σ was overexpressed in these cells by stable transfection, thereby achieving levels comparable to those observed in COLO-357 and BxPC3 cells. The overexpression of 14-3-3σ did not have a significant effect on cell cycle distribution, indicating that in these cells high levels of 14-3-3σ do not cause a G2/M cell cycle arrest. However, 14-3-3σ overexpressing clones exhibited increased resistance towards cisplatinum induced apoptosis, as evidenced by increased cell survival of the transfected clones in conjunction with a decrease in caspase-3 activation and PARP cleavage in response to cisplatinum by comparison with effects observed in sham transfected cells. Furthermore, silencing 14-3-3σ in T3M4 cells, which have high endogenous levels of this protein, sensitized these cells to cisplatinum treatment. These observations indicate that the high levels of 14-3-3σ contribute to the mechanisms that induce chemoresistance to cisplatinum in pancreatic cancer and attenuate the pro-apoptotic signaling pathway in these cells. Several additional lines of evidence support this conclusion. For example, increased 14-3-3σ was detected in drug-resistant human PDAC cell lines (Sinha, P. et al. 1999. Electrophoresis 20:2952-2960); deletion of 14-3-3σ was shown to sensitize colorectal cancer cells to doxorubicin induced apoptosis (Chan, T. A. et al. 1999. Nature 401:616-620); 14-3-3σ has been identified as one of the proteins responsible for the doxorubicin resistance in the breast cancer cell line MCF-7/AdVp3000 (Liu, Y. et al. 2006. Cancer Res. 66:3248-3255); and overexpression of 14-3-3σ in HEK293 cells enhanced resistance towards mitoxantrone (Liu, Y. et al. 2006. Cancer Res. 66:3248-3255). Taken together with the results of the experiments described herein, the data demonstrate that silencing or disrupting 14-3-3 sigma function can sensitize pancreatic cancer cells to drug induced apoptosis.

One of the hallmarks of PDAC is the ability of pancreatic cancer cells to invade and metastasize. The EGF receptor has been implicated as having an important role in many cancers, where it acts to enhance proliferation, motility, invasiveness, metastasis, and chemoresistance (Ciardello, F. and G. Tortora. 2008. NEJM 358:1160-1174; Normanno, N. et al. 2001. Front. Biosci. 6:D685-D707). Cultured human pancreatic cancer cell lines and human PDACs frequently express relatively high levels of the EGF receptor, as well as the related receptors ErbB-2/HER2 and ErbB-3/HER3 (Korc, M. 1998. Surg. Oncol. Clin. N. Am. 7:25-41). Moreover, EGF and related ligands, such as transforming growth factor-alpha (TGF-α), heparin-binding EGF like growth factor (HB-EGF), and amphiregulin enhance the proliferation and/or invasiveness of these cells (Korc, M. 1998. Surg. Oncol. Clin. N. Am. 7:25-41; Li, J. et al. 2004. Int. J. Oncol. 25:203-210), indicating that the EGF receptor participates in many aberrant autocrine and paracrine loops that may contribute to the biological aggressiveness of PDAC. The concomitant presence of the EGF receptor and either EGF or TGF-α is associated with enhanced tumor aggressiveness and shorter post-operative survival periods (Yamanaka, Y. et al. 1993. Anticancer Res. 13:565-569). The results described herein where high levels of 14-3-3σ were associated with increased basal motility and a dramatic increase in EGF-stimulated invasiveness in PANC-1 cells indicates that the concomitant overexpression of the EGF receptor and 14-3-3σ may greatly enhance the invasive capacity of pancreatic cancer cells in vivo. However, in T3M4 cells, silencing of 14-3-3σ was not associated with a change in motility, and led to increased EGF-stimulated cell invasion. By contrast, in the same cells, IGF-1-mediated invasion was robust in the presence of endogenously high levels of 14-3-3σ, and this effect was completely suppressed following 14-3-3σ silencing. Taken together, these observations indicate that the role of 14-3-3σ with respect to motility and invasion is cell type and context specific. In view of the important role of EGF in pancreatic cancer, the results described herein indicate that the concomitant targeting of EGFR and 14-3-3σ would be of therapeutic benefit in PDAC. Therefore, the present invention is also a method of treating pancreatic cancer wherein treatment targets both EGFR and 14-3-3σ.

It has also been found that the EGF-mediated increase in cancer cell invasiveness was completely blocked by LY294002, a PI3K inhibitor, by SB203580, a p38 MAPK inhibitor, and by PP2, a Src-kinase inhibitor. These data indicate that in the presence of high levels of 14-3-3σ, the PI3K, p38 MAPK and Src pathways contribute to this effect. It is known that both PI3K and p38 MAPK are activated by Src kinase upon EGFR activation (Summy, J. M. et al. 2005. Pancreas 31:263-274), and that down regulation of Src by siRNA-mediated silencing or inhibition of Src kinase activity attenuates pancreatic tumor progression and metastasis in nude mice (Trevino, J. G. et al. 2006. Am. J. Pathol. 168:962-972). In view of the known ability of 14-3-3σ to act as an adapter protein for signal transduction, the data described herein indicate that in some pancreatic cancer cells 14-3-3σ promotes more efficient interactions between EGFR and Src kinase, leading to the marked increase in EGF-mediated invasiveness. As a result, the method of treatment of the present invention involves administration of compounds that both block EGFR and inhibit activity of 14-3-3σ, either directly or indirectly.

The three pancreatic cancer cell lines that exhibited high levels of intracellular 14-3-3σ also secreted 14-3-3σ into the medium. Moreover, engineered overexpression of 14-3-3σ in PANC-1 cells was accompanied by a parallel increase in 14-3-3σ secretion from these cells. Therefore, for the first time it has been shown that 14-3-3σ is actually secreted by cancer cells. The data further demonstrated that the release of 14-3-3σ by the pancreatic cancer cells into the conditioned medium was not a spurious event caused by cell death. First, there was differential release of 14-3-3σ by the cells. Thus, detectable levels of 14-3-3σ were not observed in the medium from ASPC-1 cells in spite of the fact that they expressed 14-3-3σ, and only low levels of 14-3-3σ were present in the medium from COLO-357 cells in spite of its marked abundance in these cells. Moreover, PANC-1 cells, which expressed the lowest levels of 14-3-3σ in cell lysates, exhibited similar levels in the medium as observed in medium from COLO-357 cells, in spite of the known resistance of PANC-1 cells to apoptosis and anoikis. Second, under the experimental conditions used in the present study, the cells were viable by morphological criteria and, whenever tested, by tyrpan blue exclusion. Third, tubulin, a cytoplasmic marker, was not detectable in the medium by Western blotting, indicating that cytoplasmic proteins were not being released into the medium in a non-specific manner. Therefore, 14-3-3σ is a biomarker of cancer cells that can be exploited in a method of diagnosis of pancreatic cancer.

Previously, it was reported that 14-3-3σ is secreted by differentiated keratinocytes, and, that following release, 14-3-3σ induces MMP-1 expression in dermal fibroblasts in a co-culture system (Ghahary, A. et al. 2004. J. Invest. Dermatol. 122:1188-1197; Ghahary, A. et al. 2005. Dermatology 124:170-177). Moreover, treatment of fibroblasts with exogenous recombinant 14-3-3σ sigma led to increased MMP-1 and MMP-3 expression by fibroblasts. In view of the ability of MMP-1 to degrade fibrillar collagen and of MMP-3 to promote carcinogenesis, these data indicate that secreted 14-3-3σ may have an important role within the tumor microenvironment that may enhance cancer progression. Therefore, 14-3-3σ can serve as a serum marker for those pancreatic cancers that express the protein at high levels.

Therefore, the present invention features a composition for identifying patients with pancreatic cancer or patients with recurrence of pancreatic cancer. This composition comprises an agent that binds a pancreatic cancer-specific biomarker protein. In particular embodiments, the agent is used in a method of diagnosing pancreatic cancer by contacting a test sample from a human subject with the agent and detecting the level of binding of the test agent to the pancreatic cancer-specific biomarker protein, wherein an increase in the level of binding as compared to a level known to be seen in patients without pancreatic cancer is indicative of the presence of pancreatic cancer. In a preferred embodiment the pancreatic cancer specific biomarker protein is 14-3-3σ.

Another object of the present invention is a method of diagnosing pancreatic cancer in a patient which comprises contacting blood from a patient suspected of having pancreatic cancer or a patient that previously was diagnosed with pancreatic cancer in vitro with an agent that binds the pancreatic cancer-specific biomarker protein. In a preferred embodiment the pancreatic cancer-specific protein is 14-3-3σ.

Yet another object of the present invention is a method for treating pancreatic cancer in a patient which comprises administering to a patient with pancreatic cancer an effective amount an agent that inhibits activity of EGFR and an agent that inhibits activity of 14-3-3σ.

In the context of the present invention, “an effective amount” is defined as an amount of a compound capable of inhibiting activity of the receptor or the protein. The amount can be determined by a variety of means well known to those of skill in the art. For any compound or drug that is currently approved for use to treat pancreatic cancer by inhibiting activity of EGFR, one of skill could also determine “an effective amount” based on the approved dose ranges for the drug, doses which are commonly listed in sources such as regulatory agency approved drug labeling.

It is contemplated that one of skill in the art will choose the most appropriate animal model system to test for inhibition of activity of EGFR and 14-3-3σ depending on the type of drug product to be developed. The medical literature provides detailed disclosure on the advantages and uses of a wide variety of such models.

Once a test drug or a combination of drugs has shown to be effective in vivo in animals, clinical studies can be designed based on the doses shown to be safe and effective in animals. One of skill in the art will design such clinical studies using standard protocols as described in textbooks such as Spilker (2000. Guide to Clinical Trials. Lippincott Williams & Wilkins: Philadelphia).

Additionally, the present invention is a method of diagnosing pancreatic cancer in a patient or of identifying the recurrence of pancreatic cancer in a patient. The diagnosis can be through detection of 14-3-3σ as a protein marker in serum or blood of a patient.

In one embodiment, the presence of the pancreatic cancer-specific biomarker, the 14-3-3σ protein, is assessed by detecting the presence or level of the protein itself. Detection of a pancreatic cancer-specific biomarker involves contacting a biological sample with a compound or an agent capable of binding the biomarker.

An example of an agent for binding and detecting 14-3-3σ is an antibody capable of binding to the protein. Accordingly, the present invention also provides antibodies raised against 14-3-3σ, or portions thereof. 14-3-3σ-specific biomarker proteins can be expressed using conventional expression systems, purified and used to immunize an animal for antibody production. Antibodies can be polyclonal, or more desirably, monoclonal and can be produced by any conventional method in the art. For example, monoclonal antibodies can be prepared using any technique which provides for the production of antibody molecules by continuous cell lines in culture. These include, but are not limited to, the hybridoma technique, the human B-cell hybridoma technique, and the EBV-hybridoma technique (Kohler et al. 1975. Nature 256:495-497; Kozbor et al. 1985. J. Immunol. Methods 81:31-42; Cote et al. 1983. Proc. Natl. Acad. Sci. 80:2026-2030; Cole et al. 1984. Mol. Cell Biol. 62:109-120).

An intact antibody, antibody derivative, or a fragment thereof (e.g., Fab or F(ab′)₂) can be used. In certain embodiments, the antibody is labeled. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a protein or DNA probe with biotin such that it can be detected with fluorescently labeled streptavidin. In some embodiments, the antibody is labeled by adsorption to latex beads for use in a rapid latex agglutination assay, wherein agglutination is indicative of the presence of 14-3-3σ. Suitable antibodies useful for biomarker binding and detection can be routinely generated by the skilled artisan.

The detection methods described herein can be used to detect the presence of the biomarker protein in a biological sample in vitro as well as in vivo. In vitro techniques for detection of biomarker protein include, but are not limited to, agglutination assays, enzyme-linked immunosorbent assays (ELISAs), western blots, immunoprecipitations, and immunofluorescence assays. Alternatively, a biomarker protein can be detected in vivo in a subject by introducing into the subject a labeled antibody against the biomarker protein. For example, the antibody can be labeled with a radiotracer or radiopharmaceutical, or fluorescent or other imaging marker whose presence and location in a subject can be detected by standard imaging techniques.

The following non-limiting examples are provided to further illustrate the present invention.

### EXAMPLES

### Example 1

### Cell Culture

Human pancreatic cancer cell lines ASPC-1, PANC-1, and BxPC3 were purchased from American Type Culture Collection (Manassas, Va.). COLO-357 and T3M4 human pancreatic cancer cell lines were obtained from R. S. Metzgar (Duke University, Durham, N.C.). ASPC-1, BxPC3, and T3M4 cells were grown in RPMI, and PANC-1, COLO-357 were grown in DMEM (Mediatech, Herndon, Va.), both supplemented with 7.5-10% fetal bovine serum (FBS) (Omega Scientific, Tarzana, Calif.). Cells were cultured at 37° C. in humidified air with 5% CO₂. Serum free media used in this study refers to DMEM or RPMI supplemented with 0.1% bovine serum albumin (BSA), 5 μg/mL apo-transferrin, and 5 ng/mL sodium selenite.

### Example 2

### Immunoblotting

Immunoblotting was done as described previously (Kleeff, J. and M. Korc. 1998. J. Biol. Chem. 273:7495-7500). For the analysis of secreted 14-3-3σ protein, RPMI or DMEM supplemented with 0.05% FBS, 5 μg/mL apo-transferrin, and 5 ng/mL sodium selenite, referred to as conditioned media, was used. Conditioned media were collected 48 hours after feeding the cells. Protease inhibitor cocktail (Roche, Indianapolis, Ind.) was added to the collected media, and media were concentrated using Ultracel YM-10 membrane filter column (Millipore, Billerica, Mass.). The antibodies used in the studies were: anti-14-3-3σ that were either rabbit polyclonal from Immuno-Biological Laboratories Co. Ltd (Gunma, Japan) or mouse monoclonal from Neomarkers (Fremont, Calif.); rabbit polyclonal against anti-14-3-3η from Immuno-Biological Laboratories; rabbit polyclonal against 14-3-3β (C-20), 14-3-3ε (T-16), 14-3-3γ (C-16), 14-3-3θ (C-17), 14-3-3ζ (C-16), and Erk-2 (C-14) from Santa Cruz Biotechnology (Santa Cruz, Calif.); and rat monoclonal anti-tubulin from Abcam (Cambridge, Mass.).

### Example 3

### Immunohistochemistry

Sections (8 μm) of normal and pancreatic cancer tissue were cut from paraffin embedded tissue. The normal tissues were obtained from an organ donor program, and the cancer tissues were obtained following surgical resection of PDACs. Slides were de-paraffinized and rehydrated. The intrinsic peroxidase was blocked with 0.3% H₂O₂ in methanol for 30 minutes. Antigen demasking was done in 10 μmol/L sodium-citrate buffer (pH 6.0) by heating to near boiling in a microwave, and then slowly cooling down to 23° C. Slides were blocked with 5% goat serum in PBS for 30 minutes. Then, tissue sections were incubated in 14-3-3σ antibody diluted with 5% goat serum overnight in the cold room. Sections were then incubated for 30 minutes in biotinylated secondary antibody from Vectastain Universal kit (Vector Laboratories, Burlingame, Calif.). Finally, sections were incubated in Vectastatin ABC-Reagent and stained with DAB and lightly counterstained with Hematoxylin. Staining in the absence of the primary antibody served as a negative control and did not yield any signal.

### Example 4

### Laser Capture

Tissue specimens were obtained from 7 PDAC samples and 3 normal human pancreatic samples. The use of human tissue samples was approved by the Human Subjects Committee at Dartmouth Medical School. Laser capture microdissection (LCMD) on these samples was carried out as previously described (Ketterer, K. et al. 2003. Clin. Cancer Res. 9:5127-5136). Briefly, 6-8 μm tissue cryosections were quickly fixed in 75% ethanol and stained with 1.5% Eosin Y (Sigma, St. Louis, Mo.). Slides were air dried after dehydration in ethanol and incubation in xylene and subjected to LCMD using the Pix Cell instrument and CapSure LCM Caps (Arcturus Engineering, Mountain View, Calif.).

### Example 5

### Reverse Transcription and RT Quantitative PCR

Total RNA from each laser captured tissue sample was isolated using Absolutely RNA Nanoprep or Strataprep Total RNA Microprep kit (Stratagene, La Jolla, Calif.). The concentration of RNA was determined using RiboGreen (Molecular Probes, Eugene, Oreg.) and a CytoFluor florescence plate reader (Per-Septive Biosystems, Framingham, Mass.). RNA (20 ng) was reverse transcribed using Sensiscript RT kit (Qiagen, Valencia, Calif.). For studies in cell lines, total RNA was extracted by the acid guanidinium thiocyanate-phenol-chloroform method (Chomczynski, P. and N. Sacchi. 1987. Anal. Biochem. 162:156-159). Integrity of RNA was assessed by running on a 1% agarose gel, staining with ethidium bromide, and visualizing under UV light. RNA was reverse transcribed using random hexamer primers and the SuperScript kit (Invitrogen, Carlsbad, Calif.) according to the manufacturer's protocol. For tissue and cell line studies, fifty cycles of quantitative real-time PCR (Q-PCR) was performed in a 7700 sequence detector from Applied Biosystems (Foster City, Calif.). Primers and probe sets were designed using Primer Express 1.5 software (Applied Biosystems). Primers and probes were verified for specificity by individually blasting them against the NCBI database, and custom ordered from Qiagen. The following sequences were used for the primers and probes: 14-3-3β forward: AAGAGAGAAATCTGCTCTCTGTTGC (SEQ ID NO:1), 14-3-3β reverse: GGAGATGACACGCCAGGAAG (SEQ ID NO:2), 14-3-3β probe: 6-FAM-ACAAGAATGTGGTAGGCGCCCGC-TAMRA (SEQ ID NO:3); 14-3-3ε forward: ATGGATGATCGAGAGGATCTGG (SEQ ID NO:4), 14-3-3ε reverse: CTCCACCATTTCGTCGTATCG (SEQ ID NO:5), 14-3-3ε probe: 6-FAM-CAGGCGAAGCTGGCCGAGCA-TAMRA (SEQ ID NO:6); 14-3-3γ forward: CGTGCGTACCGGGAGAAG (SEQ ID NO:7), 14-3-3γ reverse: TCCAGCAGGCTCAGCACA (SEQ ID NO:8), 14-3-3γ probe: 6-FAM-AGAGAAGGAGTTGGAGGCTGTGTGCCA-TAMRA (SEQ ID NO:9); 14-3-3η forward: TGGTGCCAGGCGATCTTC (SEQ ID NO:10), 14-3-3η reverse: TCGTTTCCATCAGCCATGG (SEQ ID NO:11), 14-3-3η probe: 6-FAM-TGGAGGGTCATTAGCAGCATTGAGCAG-TAMRA (SEQ ID NO:12); 14-3-3σ forward: CAGTCTGATCCAGAAGGCCAA (SEQ ID NO:13), 14-3-3σ reverse: GAAGGCTGCCATGTCCTCA (SEQ ID NO:14), 14-3-3σ probe: 6-FAM-TGGCAGAGCAGGCCGAACGC-TAMRA (SEQ ID NO:15); 14-3-3θ forward: CGCCTGGAGGGTCATCTCTA (SEQ ID NO:16), 14-3-3θ reverse: TCCTTAATCAGCTGCAACTTCTTG (SEQ ID NO:17), 14-3-3θ probe: 6-FAM-TCGAGCAGAAGACCGACACCTCCG-TAMRA (SEQ ID NO:18); 14-3-3ζ forward: GAGCAAGGAGCTGAATTATCC (SEQ ID NO:19), 14-3-3ζ reverse: GACCTACGGGCTCCTACAAC (SEQ ID NO:20), 14-3-3ζ probe: 6-FAM-TGAGGAGAGGAATCTTCTCTCAGTTGCT-TAMRA (SEQ ID NO:21).

### Example 6

### Cloning

Total RNA from BxPC3 was reverse transcribed using random hexamer primers as described above, and 14-3-3σ cDNA was amplified by PCR using the following primers: forward, ATAAGCTTCCAGAGCCATGGAGA (SEQ ID NO:22); reverse, CACGTGGCTCTGGGGCTCCTG (SEQ ID NO:23). The PCR amplified gene product was cloned into TOPO-TA cloning vector (Invitrogen). Then the gene was cloned in the HindIII/BbrpI sites of the pMH vector (Roche Biochemicals, Indianapolis, Ind.), which encodes a c-terminal hemagglutinin-antigen (HA) epitope-tag in frame with the gene (construct named pMH-14-3-3σ-HA). PANC-1 cells were stably transfected with pMH-14-3-3σ-HA. The stable clones were selected in complete DMEM medium supplemented with 800 μg/mL of Geneticin (Gibco laboratories, Grand Island, N.Y.). Two clones expressing similar levels of 14-3-3σ were chosen for subsequent experiments.

### Example 7

### Immunocytofluorescence

PANC-1 sham and 14-3-3σ-transfected clones (1.5×10⁵) were seeded on LabTek chamber slides (Nalge Nunc, Rochester, N.Y.) and incubated at 37° C. for 48 hours, washed with PBS, and fixed in 2% paraformaldehyde for 10 minutes. Cells were washed extensively with PBS, incubated for 10 minutes with 50 μmol/L NH₄Cl in PBS, and permeabilized/blocked with 0.1% triton x-100/PBS-2% BSA for 15 minutes (21). Cells were then incubated for 1 hour at 23° C. with anti-14-3-3σ antibody, washed with PBS, and then incubated with Alexa-Fluor 488 goat anti-mouse secondary antibody (Invitrogen) for 30 minutes at 23° C. Finally, cells were counterstained with Hoechst 33258 (0.5 mL of 0.2 μg/mL stock) for 5 minutes. Cells were examined using Olympus BX60 upright microscope (Olympus, Tokyo, Japan) fitted with Olympus DP 70 camera, and pictures were taken using Image-Pro plus software (MediaCybernetics, Silver Spring, Md.).

### Example 8

### MTT Assay

Sham-transfected PANC-1 cells and 14-3-3σ transfected clones (1×10⁴) were plated in 96-well plates (six wells per sample). Cells were allowed to attach and grow for 48 hours in complete medium, and then treated with complete medium in the absence or presence of 10 μg/mL of cisplatinum (Sigma) for 48 hours, followed by the addition of MTT (3-[4,5-dimethylthiazol-2-yl]-2,5-diphenyltetrazolium bromide) (Sigma) at a final concentration 0.55 μg/mL. After an additional 4 hour incubation, absorbance at 570 nm was determined, with a reference of 650 nm, using a Emax precision microplate reader (Molecular devices, Sunnyvale, Calif.), as previously reported (Arnold, N. B. et al. 2004. Cancer Res. 64:3599-3606). It was previously determined that in pancreatic cancer cell lines the MTT assay correlates closely with cell growth as determined by cell counting and by assessing [³H]thymidine incorporation (Raitano, A. B. and M. Korc. 1990. J. Biol. Chem. 265:10466-10472; Baldwin, R. L. and M. Korc. 1993. Growth Fact. 8:23-34).

### Example 9

### Cisplatinum Induced Apoptosis

Sham or 14-3-3σ transfected PANC-1 cells were incubated for 24 hours in complete medium in the absence or presence of 10 μg/mL of cisplatinum. Scrambled or 14-3-3σ specific shRNA infected T3M4 cells were incubated in complete medium for 24 hours that was supplemented with 10 μg/mL cisplatinum for the initial 2 or 6 hours, or the entire 24 hours. Both floating and adherent cells were then collected and lysed in the buffer containing protease inhibitors, subjected to 10% SDS-PAGE, and immunoblotted with specific antibodies against cleaved Poly (ADP-ribose) Polymerase (PARP) and cleaved caspase-3 (Cell Signaling Technology, Danvers, Mass.).

### Example 10

### Cell Migration Assay

To assess the motility properties, in vitro wound-healing assays or migration assays were performed. Cells were grown to confluence in 6-well tissue culture plates, serum starved overnight, and the monolayer was scratched with a 200 μL pipette tip generating two parallel wounds. The scratched wells were washed to remove cell debris and incubated for the next 24 hours in serum free media in the absence or presence 1 nmol/L EGF with or without the Src family kinase inhibitor PP2 (4-amino-5-(4-chlorophenyl)-7-(t-butyl)pyrazolo[3,4-d]pyrimidine) (Alexis, La Jolla, Calif.). To monitor cell migration, photographs were taken of each well at five marked locations under 40× magnification immediately after wounding (t0) and again 24 hours after wounding (t24), using an inverted microscope fitted with a digital camera (Nikon Diaphot 300, Nikon, Japan). The wound area of five matched pictures at t0 and t24 from each well were then measured using the ImageJ software (NIH, Bethedsa, Md.), and the percentage change in the wound area was calculated.

### Example 11

### Cell Invasion Assay

Cell invasion was measured as reported previously, with some modification (Rowland-Goldsmith, M. A. et al. 2001. Clin. Cancer Res. 7:2931-2940). Briefly, cells were suspended in 500 μL of serum-free medium (0.1% BSA) and placed onto the upper compartment of Matrigel-coated transwell chambers (8 μm pore sized, BioCoat Matrigel Invasion Chambers, BD Biosciences, San Jose, Calif.). The lower compartment was filled with 750 μL of medium containing serum free media in the absence or presence of the respective growth factors. After 18-20 hours, cells on the upper surface of the filter were carefully removed with a cotton swab and the membranes were fixed in methanol. The cells that had migrated through the membrane to the lower surface of the filter were stained with toluidine blue (Fisher Scientific, Fair Lawn, N.J.) and counted using a light microscope.

### Example 12

### Downregulation of 14-3-3σ by shRNA Using Lentivirus Gene Delivery System

Two different shRNA oligo sequences targeting 14-3-3σ (Genebank accession no. NM_{—}006142) were designed based on siRNA sequences (underlined) against 14-3-3σ from Dharmacon.

sh1
(SEQ ID NO: 24)
sense: TCGAGACAACCTGACACTGTTTCAAGAGAACAGTGTCAGGTT
GTCTCGTTTTTTC;
sh1
(SEQ ID NO: 25)
antisense: TCGAGAAAAAACGAGACAACCTGACACTGTTCTCTTGA
AACAGTGTCAGGTTGTCTCGA;
sh2
(SEQ ID NO: 26)
sense: TGGAGAGAGCCAGTCTGATCTTCAAGAGAGATCAGACTGGCT
CTCTCCTTTTTTC;
sh2
(SEQ ID NO: 27)
antisense: TCGAGAAAAAAGGAGAGAGCCAGTCTGATCTCTCTTGA
AGATCAGACTGGCTCTCTCCA.

The control shRNA was designed not to target any known mammalian gene by scrambling siRNA sequence used in sh2 (underlined).

shCtrl
(SEQ ID NO: 28)
sense: TACGAGCGAGGTGCCGATATTTCAAGAGAATATCGGCACCTC
GCTCGTTTTTTTC;
shCtrl
(SEQ ID NO: 29)
antisense: TCGAGAAAAAAACGAGCGAGGTGCCGATATTCTCTTGA
AATATCGGCACCTCGCTCGTA.

The sense and anti-sense oligonucleotide templates (Sigma Genosys) were annealed together, and cloned in the XhoI/HpaI sites of the pLentiLox 3.7 lentiviral vector (Addgene, Cambridge, Mass.). Lentivirus particles were produced by four plasmid transfection system as follows. 293T cells were transfected with 60 μg of shRNA lentiviral vector construct and 30 μg each of the helper plasmids (VSVG, pRSV-Rev, and pMDLg/pRRE; all from Addgene). The supernatant containing the virus particles was collected at 48 and 72 hours post-transfection and concentrated using an ultracentrifuge at 25,000 rpm for 1.5 hours. The pellets were suspended in 25 μL of Opti-Mem media (Invitrogen) overnight, pooled together, and supernatant were aliquoted. Viral titer was determined by generating a 10-fold dilution series and infecting 293T cells (1×10⁵/well) in a 6-well plate, and by analyzing the percent EGFP positive cells by flow cytometry at 72 hours after infection. T3M4 cells were infected at a multiplicity of infection (MOI) of 40 and the knockdown of 14-3-3σ was confirmed by western blotting and Q-PCR.

What is claimed is:

1. A composition for diagnosing pancreatic cancer comprising an agent that binds a pancreatic cancer-specific biomarker protein.

2. The composition of claim 1, wherein said agent binds 14-3-3σ.

3. A method of diagnosing pancreatic cancer comprising contacting a test sample from a human subject with an agent of claim 1 and detecting the level of binding of the test agent to 14-3-3σ, wherein an increase in the level of binding as compared to the level seen in humans known to cancer-free is indicative of pancreatic cancer.

4. A method of treating pancreatic cancer comprising administering to a patient with pancreatic cancer an effective amount of a compound that inhibits activity of EGFR and a compound that inhibits activity of 14-3-3σ.


SEQUENCE LISTING (29 sequences)

SEQ ID NO: 1 - DNA, 25 bp, Artificial Sequence
  Synthetic oligonucleotide
aagagagaaatctgctctctgttgc

SEQ ID NO: 2 - DNA, 20 bp, Artificial Sequence
  Synthetic oligonucleotide
ggagatgacacgccaggaag

SEQ ID NO: 3 - DNA, 23 bp, Artificial Sequence
  Synthetic oligonucleotide
acaagaatgtggtaggcgcccgc

SEQ ID NO: 4 - DNA, 22 bp, Artificial Sequence
  Synthetic oligonucleotide
atggatgatcgagaggatctgg

SEQ ID NO: 5 - DNA, 21 bp, Artificial Sequence
  Synthetic oligonucleotide
ctccaccatttcgtcgtatcg

SEQ ID NO: 6 - DNA, 20 bp, Artificial Sequence
  Synthetic oligonucleotide
caggcgaagctggccgagca

SEQ ID NO: 7 - DNA, 18 bp, Artificial Sequence
  Synthetic oligonucleotide
cgtgcgtaccgggagaag

SEQ ID NO: 8 - DNA, 18 bp, Artificial Sequence
  Synthetic oligonucleotide
tccagcaggctcagcaca

SEQ ID NO: 9 - DNA, 27 bp, Artificial Sequence
  Synthetic oligonucleotide
agagaaggagttggaggctgtgtgcca

SEQ ID NO: 10 - DNA, 18 bp, Artificial Sequence
  Synthetic oligonucleotide
tggtgccaggcgatcttc

SEQ ID NO: 11 - DNA, 19 bp, Artificial Sequence
  Synthetic oligonucleotide
tcgtttccatcagccatgg

SEQ ID NO: 12 - DNA, 27 bp, Artificial Sequence
  Synthetic oligonucleotide
tggagggtcattagcagcattgagcag

SEQ ID NO: 13 - DNA, 21 bp, Artificial Sequence
  Synthetic oligonucleotide
cagtctgatccagaaggccaa

SEQ ID NO: 14 - DNA, 19 bp, Artificial Sequence
  Synthetic oligonucleotide
gaaggctgccatgtcctca

SEQ ID NO: 15 - DNA, 20 bp, Artificial Sequence
  Synthetic oligonucleotide
tggcagagcaggccgaacgc

SEQ ID NO: 16 - DNA, 20 bp, Artificial Sequence
  Synthetic oligonucleotide
cgcctggagggtcatctcta

SEQ ID NO: 17 - DNA, 24 bp, Artificial Sequence
  Synthetic oligonucleotide
tccttaatcagctgcaacttcttg

SEQ ID NO: 18 - DNA, 24 bp, Artificial Sequence
  Synthetic oligonucleotide
tcgagcagaagaccgacacctccg

SEQ ID NO: 19 - DNA, 21 bp, Artificial Sequence
  Synthetic oligonucleotide
gagcaaggagctgaattatcc

SEQ ID NO: 20 - DNA, 20 bp, Artificial Sequence
  Synthetic oligonucleotide
gacctacgggctcctacaac

SEQ ID NO: 21 - DNA, 28 bp, Artificial Sequence
  Synthetic oligonucleotide
tgaggagaggaatcttctctcagttgct

SEQ ID NO: 22 - DNA, 23 bp, Artificial Sequence
  Synthetic oligonucleotide
ataagcttccagagccatggaga

SEQ ID NO: 23 - DNA, 21 bp, Artificial Sequence
  Synthetic oligonucleotide
cacgtggctctggggctcctg

SEQ ID NO: 24 - DNA, 55 bp, Artificial Sequence
  Synthetic oligonucleotide
tcgagacaacctgacactgtttcaagagaacagtgtcaggttgtctcgttttttc

SEQ ID NO: 25 - DNA, 59 bp, Artificial Sequence
  Synthetic oligonucleotide
tcgagaaaaaacgagacaacctgacactgttctcttgaaacagtgtcaggttgtctcga

SEQ ID NO: 26 - DNA, 55 bp, Artificial Sequence
  Synthetic oligonucleotide
tggagagagccagtctgatcttcaagagagatcagactggctctctccttttttc

SEQ ID NO: 27 - DNA, 59 bp, Artificial Sequence
  Synthetic oligonucleotide
tcgagaaaaaaggagagagccagtctgatctctcttgaagatcagactggctctctcca

SEQ ID NO: 28 - DNA, 55 bp, Artificial Sequence
  Synthetic oligonucleotide
tacgagcgaggtgccgatatttcaagagaatatcggcacctcgctcgtttttttc

SEQ ID NO: 29 - DNA, 59 bp, Artificial Sequence
  Synthetic oligonucleotide
tcgagaaaaaaacgagcgaggtgccgatattctcttgaaatatcggcacctcgctcgta